(19) United States
(12) Patent Application Publication
(10) Pub. No.: US 2019/0175355 A1
(43) Pub. Date: Jun. 13, 2019

(54) SHOULDER PROSTHESIS COMPONENT, SUCH AS A HUMERAL COMPONENT OR A GLENOID COMPONENT, FOR A PATIENT, AND METHOD FOR PRODUCING SUCH A SHOULDER PROSTHESIS COMPONENT FOR A PATIENT

(71) Applicant: Tornier, Montbonnot-Saint-Martin (FR)

(72) Inventors: François Boux de Casson, Le Sappey en Chartreuse (FR); Jean-Emmanuel Cardon, Domene (FR); Florian Laboulfie, Meylan (FR); Nicolas Renaud Niechel, Le Sappey en Chartreuse (FR)

(21) Appl. No.: 16/216,134

(22) Filed: Dec. 11, 2018

(30) Foreign Application Priority Data
Dec. 12, 2017 (EP) 17306747.1

(51) Int. Cl. A61F 2/40; A61F 2/28; A61F 2/30

(52) U.S. Cl. CPC A61F 2/4081; A61F 2/28; A61L 27/3608; A61F 2/30942; A61F 2002/30948; A61F 2/4059

(57) ABSTRACT

The shoulder prosthesis component comprises an implantation body, which defines an implantation axis and which is designed to be received within a bone of a patient that includes a cortical bone external layer and a cancellous bone internal layer, and to compact therein the cancellous bone internal layer in a planned manner that is differentiated along and around the implantation axis depending on preoperative local bone density and quantity of the cancellous bone internal layer. A method for producing such a shoulder prosthesis component for a patient is also proposed.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 C.F.R. § 1.57.

### BACKGROUND OF THE INVENTION

### Field of the Invention

The present invention relates to a shoulder prosthesis component for a patient. The invention also relates to a method for producing a shoulder prosthesis component for a patient.

### Description of the Related Art

In a healthy human shoulder, the head of the humerus, which is generally ball-shaped, and the glenoid cavity of the scapula articulate with each other and form a ball-and-socket joint. Total shoulder arthroplasty is a common treatment for shoulder pain resulting from arthritis or injury and leads to replace the ball-and-socket joint by a shoulder orthopedic prosthesis.

Such a shoulder prosthesis generally includes both a glenoid component to be fixedly implanted on the glenoid of the scapula and a humeral component to be fixedly implanted on the humerus, the glenoid component and the humeral component being provided to articulate directly with each other.

### SUMMARY OF THE INVENTION

Over the last few years, it is proposed to design the humeral and glenoid components specifically to the patient to be treated. For this purpose, images of the humerus and of the glenoid are first acquired and the side of the humeral or glenoid component, intended to be applied directly on the humerus or glenoid is then designed to match accurate bone anatomy of the humerus or glenoid of the patient: in other words, the aforesaid side of the shoulder prosthesis component is the negative image of the humerus or glenoid. In that way, the shoulder prosthesis component is implanted without removing bone material from the humerus or glenoid, which enables to optimize preservation of the osseous capital for the patient in question. However, the geometrical congruence between the shoulder prosthesis component and the humerus or glenoid may lead to devitalize at least some parts of the bone material covered by the component because of insufficient mechanical stress on these parts. In other words, this technique based on the negative image of the humerus or glenoid may induce osteolysis for the humerus or glenoid. Besides, some securement problems may also occur when the bone material that is complementary covered by the component has a poor quality.

One of the goals of the invention is to propose an improved shoulder prosthesis component for a patient, which in particular is lifelong and stable on the humerus or glenoid after implantation.

To this end, one object of the invention is a shoulder prosthesis component for a patient, comprising an implantation body, which defines an implantation axis and which is designed to be received within a bone of the patient that includes a cortical bone external layer and a cancellous bone internal layer, and to compact therein the cancellous bone internal layer in a planned manner that is differentiated along and around the implantation axis depending on preoperative local bone density and quantity of the cancellous bone internal layer.

Another object of the invention is a method for producing a shoulder prosthesis component for a patient, especially for producing the shoulder prosthesis component as defined above, this method comprising:

a data providing step, in which a bone of the patient that includes a cortical bone external layer and a cancellous bone internal layer is characterized by patient bone data representative of preoperative local bone density and quantity of the cancellous bone internal layer,

a design step, in which an implantation body of the shoulder prosthesis component to be produced is designed from the patient bone data so that the bone as prosthesized with the shoulder prosthesis component to be produced receives inwardly the implantation body and has its cancellous bone internal layer that is compacted in a planned manner that is differentiated along and around an implantation axis of the implantation body depending on preoperative local bone density and quantity of the cancellous bone internal layer, and

a manufacturing step, in which the implantation body designed at the design step is manufactured.

After implantation of the shoulder prosthesis component according to the invention on a bone of a patient, especially on a humerus or a glenoid of the patient, the cancellous bone internal layer of this bone is compacted within the bone by the implantation body of the component in a manner that is differentiated along and around a longitudinal axis of the bone. Furthermore, the manner according to which the cancellous bone internal layer is compacted is planned in function of preoperative local bone density and quantity of the cancellous bone internal layer. In that way, the implantation body of the component according to the invention is designed to compact, in other words is designed to deform the cancellous bone internal layer so as to produce a predicted result for the compaction of the bone material in the bone when the component is inserted into this bone. Thanks to the invention, an appropriate mechanical stress having a predetermined value that is both non zero and not too high can be applied by the implantation body of the component on the bone material in the bone, being noted that the predetermined value of this mechanical stress may be differentiated along and/or around the longitudinal axis of the bone according to the aforesaid planned manner: this mechanical stress stimulates the bone material in the bone and thus avoids any devitalization or even osteolysis. Furthermore, even if the cancellous bone internal layer has a poor quality, the invention enables to take this into account for designing the implantation body of the component, especially by adjusting the localization and amount of the compression performed by the implantation body, and thus to avoid any lack of securement for the implantation body into the bone.

According to additional advantageous features for the shoulder prosthesis component according to the invention:

The implantation body includes a peripheral part that is shaped and structured to compact the cancellous bone internal layer in said planned manner.

The peripheral part is also shaped and structured to be deformed while compacting the cancellous bone internal layer in said planned manner.

The peripheral part comprises a porous structure which has a porosity that is varied along and around the implantation axis depending on preoperative local bone density and quantity of the cancellous bone internal layer.

The implantation body further includes a central core that is more rigid than the peripheral part.

The peripheral part comprises an elastic shell, which is supported by the central core by means of spacers and which has a deformation capability that is varied along and around the implantation axis depending on preoperative local bone density and quantity of the cancellous bone internal layer.

The peripheral part comprises:
an intermediate, soft undercoat, which covers the central core and which has a shape adaptability that is implemented by deformation and that is varied along and around the implantation axis depending on preoperative local bone density and quantity of the cancellous bone internal layer, and
an external, frangible coating, which overlays the undercoat and which has a shape adaptability that is implemented by breaking and that is varied along and around the implantation axis depending on preoperative local bone density and quantity of the cancellous bone internal layer.

The shoulder prosthesis component further comprises an articulation member, which is provided with an articulation surface and which is provided to be coupled with the implantation body.

The shoulder prosthesis component is a humeral component, the implantation body being designed for the humerus of the patient.

The shoulder prosthesis component is a glenoid component, the implantation body being designed for the glenoid of the patient.

According to additional advantageous features for the producing method according to the invention:

The design step includes:
defining rules which are based on the patient bone data and which relate preoperative local bone density and quantity of the cancellous bone internal layer to a desired compacted condition for the cancellous bone internal layer of the bone as prosthesized with the shoulder prosthesis component to be produced, and
using the rules to design the implantation body.

The data providing step includes performing a CT scan of the bone and segmenting CT images of the CT scan into the patient bone data.

The manufacturing step includes using an additive manufacturing process.

The method further comprises coupling the implantation body manufactured at the manufacturing step with an articulation member of the shoulder prosthesis component, the articulation member being provided with an articulation surface.

### BRIEF DESCRIPTION OF THE DRAWINGS

Embodiments of the invention will be better understood from reading the description which will follow, which is given solely by way of example and with reference to the drawings, in which:

FIG. 1 is a schematic view of a humerus of a patient;

FIG. 2 is a schematic view of a humeral component that is implanted at the humerus of FIG. 1;

The FIGS. 3 and 4 are views similar to FIG. 2, respectively illustrating two other humeral components; and

FIG. 5 is a view similar to FIG. 2, illustrating a glenoid component that is implanted at a glenoid of a patient.

### DETAILED DESCRIPTION OF THE PREFERRED EMBODIMENT

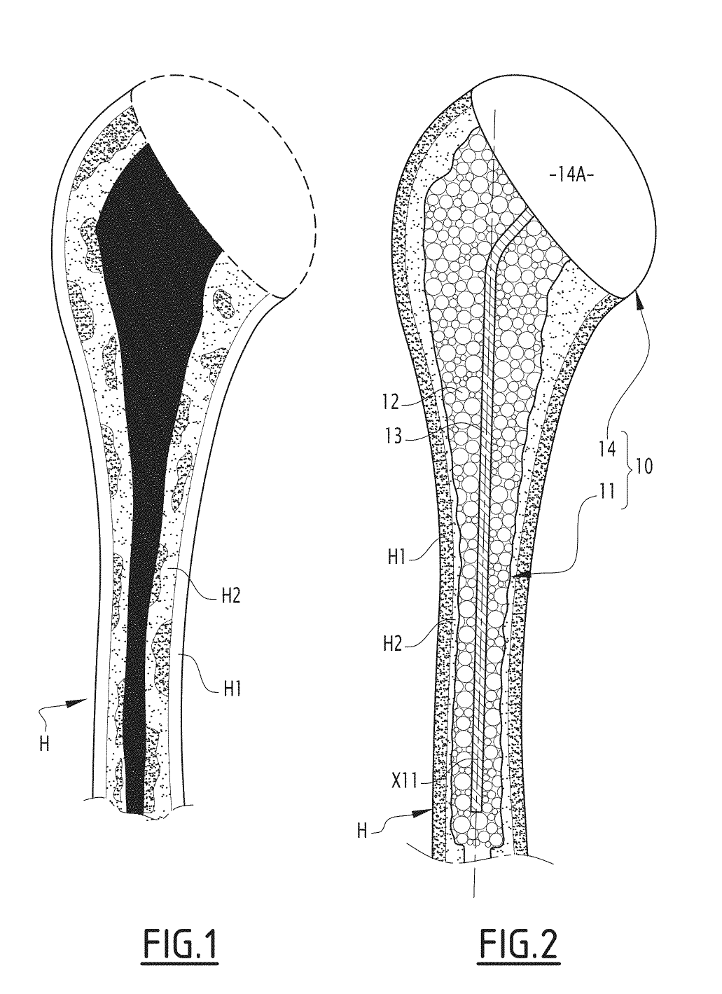

FIG. 1 shows a human humerus H intended to be prosthesized with a humeral component. More precisely, in FIG. 1 the humerus H is depicted as this humerus appears in one of the CT images of a CT scan performed on the humerus of a patient before implanting the aforesaid humeral component. The CT image corresponding to FIG. 1 allows distinguishing two different bone layers of the humerus H, especially for the diaphyseal and metaphyseal parts of the latter, i.e.:

an external bone layer H1 that is made of hard bone and that corresponds to a cortical bone layer, and

an internal bone layer H2 that is made of soft bone and that corresponds to a cancellous or spongious bone layer.

The cortical bone external layer H1 forms an outer sleeve for the humerus H, especially for the diaphyseal and metaphyseal parts of the humerus, and is internally covered by the cancellous bone internal layer H2 which in turn internally defines a free volume that corresponds to a medullary canal of the humerus H. It is important to note that the accurate shape of the cortical bone external layer H1 and the accurate shape of the cancellous bone internal layer H2 are specific to the patient to be treated. In particular, the cancellous bone internal layer H2 has bone density and quantity which vary both along and around a longitudinal axis of the humerus H, the corresponding local variations of bone density and quantity of the cancellous bone internal layer H2 being specific to the patient in question. For example, the cancellous internal bone layer H2 may include a first portion, which is located both at a first level along the longitudinal axis of the humerus H and at a first angle around this longitudinal axis and which has a bone density and a bone quantity, i.e. a bone thickness, that are different from the bone density and thickness of a second portion of the cancellous bone internal layer H2, which is located both at a second level along the longitudinal axis of the humerus and at a second angle around this longitudinal axis, the second level being different from the first level and/or the second angle being different from the first angle, it being understood that the first and second levels and the first and second angles have any respective values depending on the patient.

In view of producing a humeral component to be implanted at the humerus H of the patient in question, the local bone density and quantity of the cancellous bone internal layer H2 of the humerus H are quantified, especially for the diaphyseal part and for the metaphyseal part of the humerus. As the head of the humerus H is intended to be at least partly removed during surgery, the bone anatomy of this head has not be characterized, which explains why the head of the humerus H is drawn only in dotted lines in FIG. 1. In practice, the technique used to perform such a quantification of bone density and quantity of the cancellous bone internal layer H2 of the humerus H is not limitative provided that this technique provides patient bone data representative of preoperative local bone density and quantity of the cancellous bone internal layer H2.

In some embodiments, these patient bone data result from segmenting CT images of a CT scan performed on the humerus H. As indicated above, an example of such a CT image is shown in FIG. 1. In such CT images, the bone density may be characterized in Hounsfield units (HU), as well known in the field of medical imaging technologies: in the example of FIG. 1, the part of the humerus H having the highest bone density, in other words the part of the humerus having a value in Hounsfield units that is higher than a threshold, appears as white and corresponds to the cortical bone external layer H1; the part of the humerus having a zero bone density appears as black and corresponds to the medullary canal of the humerus H; the other portions of the humerus H appear as darker or lighter grey and correspond to the cancellous bone internal layer H2 which has bone density whose local values are both greater than zero and lower than the bone density value of the cortical bone external layer H1. Furthermore, in such CT images, the bone quantity may be characterized in dimension units, such as millimeters, used to quantify the thickness of the cancellous bone internal layer H2. Of course, rather than considering two dimensional CT images as the one shown in FIG. 1, three dimensional CT images may be considered, on the understanding that using and segmenting such two dimensional images and/or three dimensional CT images depend on capability of the imaging installation, especially the software and hardware thereof, that is used.

In some embodiments, other imaging techniques may be used as an alternative or supplement to a CT scan in order to provide the aforesaid patient bone data. For example, X-ray images and/or MRI images may be performed on the humerus H and analyzed, typically by computer, in order to provide the aforesaid patient bone data. In any case, these data are preoperative, that is to say representative of the bone anatomy of the humerus H of the patient before this humerus is surgically treated in view of receiving a humeral component.

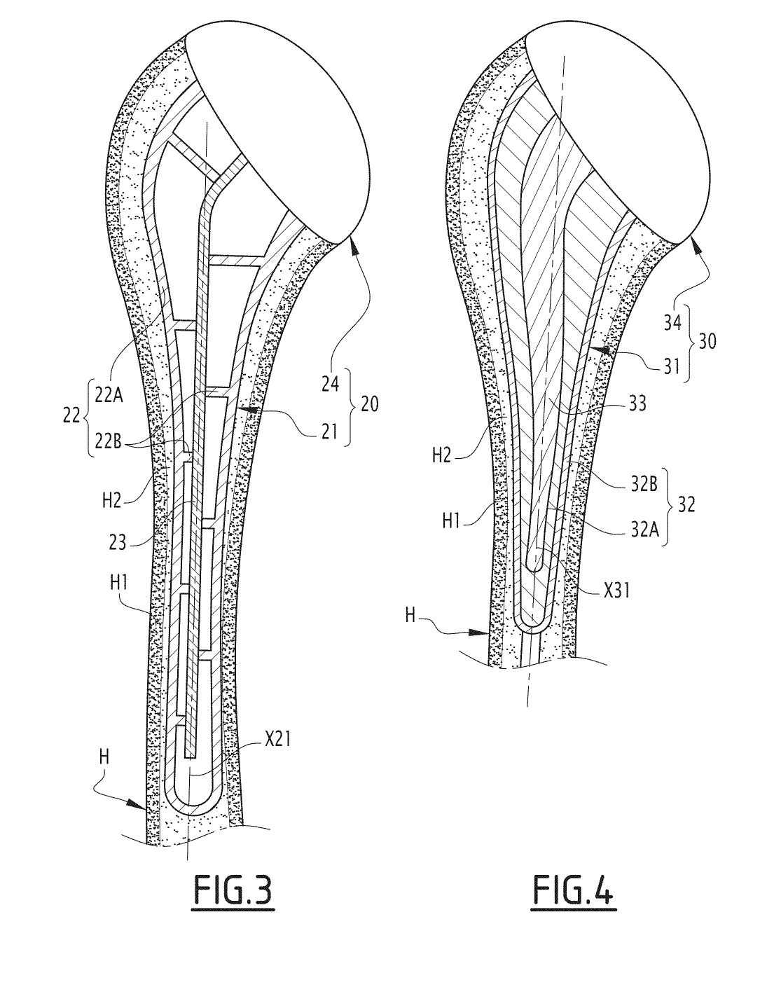

Also before surgically treating the humerus H, the aforesaid patient bone data are used to design an implantation body of a humeral component that is to be produced and intended to be implanted at the humerus during a future surgery. As explained later in reference to the FIGS. 2 to 4, various embodiments are possible for this implantation body. In any case, the design of this implantation body is based on the aforesaid patient bone data so that the humerus H as prospectively prosthesized with the humeral component to be produced receives inwardly the implantation body and has its cancellous bone internal layer H2 that is compacted in a planned manner that is differentiated along and around an implantation axis of the implantation body depending on preoperative local bone density and quantity of this cancellous bone internal layer H2.

In practice, one possibility is to use rules to design this implantation body, these rules being based on the patient bone data and relating preoperative local bone density and quantity of the cancellous bone internal layer H2 to a desired compacted condition for this cancellous bone internal layer of the humerus H as prosthesized with the humeral component to be produced. The particulars of such rules are not limitative. For example, one of these rules is to plan that a portion of the cancellous bone internal layer H2, having a low density and a high thickness has to be compacted with a high rate of compaction, for example around 50%. Another rule may be to plan that a portion of this cancellous internal bone layer H2, having a low bone density and a small thickness has to be compacted with a low rate of compaction, for example less than 50%, such as around 25%. Other rules may be contemplated. Besides, instead of using rules with compaction rates, the rules may be with compaction values.

Other possibilities for operating the design of the implantation body on the basis of the aforesaid patient bone data may be considered. More generally, it is important to understand that the implantation body is designed to apply stresses on the cancellous bone internal layer H2 and thus to deform and compact this layer H2 so as to produce a predicted result for the compaction of the various portions of that bone material in the humerus H when in the future this implantation body is inserted into the humerus. In some embodiments, designing the implantation body provides to design a peripheral part of this implantation body so that this peripheral part is shaped and structured in order to compact the cancellous internal bone layer H2 in the aforesaid planned manner. In some embodiments, the compaction effect provided by such a peripheral part of the implantation body may also take into account the fact that this peripheral part is deformed while compacting the cancellous bone internal layer H2 in the aforesaid planned manner, the implantation body being designed so that its peripheral part is shaped and structured accordingly.

Once the design of the implantation body of the humeral component to be produced is established, a following step is to manufacture this designed implantation body. The manufacturing techniques which can be used are not limitative. In some embodiments, an additive manufacturing process may be used.

Turning now to FIG. 2, a humeral component 10 is considered as a first embodiment of a humeral component produced by the method described up to here.

The humeral component 10 comprises an implantation body 11 that is designed as described above. The implantation body 11 defines an implantation axis X11 intended to be aligned with the longitudinal axis of the humerus H upon implantation of the humeral component 10 at the humerus H. As discussed above in connection with designing an implantation body on the basis of the aforesaid patient bone data representative of preoperative local bone density and quantity of the cancellous bone internal layer H2, the implantation body 11 of the humeral component 10 is designed to be received within the humerus H and to compact therein the cancellous bone internal layer H2 in a planned manner that is differentiated along and around the implantation axis X11 depending on preoperative local bone density and quantity of this cancellous bone internal layer H2: as shown in FIG. 2 in which the humerus is drawn as prosthesized by the humeral component 10, the implantation body 11 is received within the humerus H and the cancellous bone internal layer H2 is compacted by the implantation body 11 in the aforesaid planned manner. For example, one or more portions of the cancellous bone internal layer H2 are compacted with a compaction rate of around 50% (or with a given compaction value) and/or one or more other portions of this layer H2 are compacted with a higher compaction rate (or with a higher compaction value) and/or one or more other portions of this layer H2 are compacted with a lower compaction rate (or with a lower compaction value), depending on the preoperative bone density and quantity of each of these various portions that are located at different respective levels along the implantation axis X11 and/or at different respective angles around the implantation axis X11.

In some embodiments as the one shown in FIG. 2, the implantation body 11 includes a peripheral part 12 and a central core 13. The central core 13, which may be a metallic rod for example, is more rigid than the peripheral part 12, so as to support this peripheral part 12. Upon implantation of the humeral component 10, the peripheral part 12 is intended to be in contact with the cancellous bone internal layer H2 and is accordingly shaped and structured to compact this layer H2 in the aforesaid planned manner. In practice, the peripheral part 12 may be shaped and structured to be deformed while compacting the cancellous bone internal layer H2 in the aforesaid planned manner. In the example shown in FIG. 2, the peripheral part 12 comprises or even is made of a porous structure for that purpose: this porous structure is provided to have a porosity that is varied along and around the implantation axis X11 depending on preoperative local bone density and quantity of the cancellous bone internal layer H2. In that way, upon implantation of the implantation body 11 in the humerus H, the porous structure of the peripheral part 12 combines a mechanical stiffness for pressing the layer H2 and a deformation capacity for adapting its shape and thus avoiding any overstress on the layer H2: in doing so, the porous structure of the peripheral part 12 can compact the cancellous bone internal layer H2 with a predicted compaction rate that is varied along and around the implantation axis X11 in accordance with the aforesaid planned manner with which the various portions of the layer H2 are desired to be compacted. In practice, such a porous structure of the peripheral part 12 may be manufactured by additive manufacturing processes.

In some embodiments as the one shown in FIG. 2, the humeral component 10 further comprises an articulation head 14. The articulation head 14 is provided with an articulation surface 14A intended to articulate either with a glenoid prosthetic component implanted at the glenoid of the patient or directly with the glenoid of the patient, in both cases so as to restore shoulder joint for the patient. The articulation head 14 is also provided to be coupled with the implantation body 11, in particular with the central core 13 of the latter: in practice, the corresponding coupling arrangements are not limitative and will not be further described insofar as various solutions are available in the art. The other particulars of the articulation head 14 are not limitative.

Turing now to FIG. 3, a humeral component 20 is considered as a second embodiment of a humeral component produced by the method described above.

The humeral component 20 comprises an implantation body 21 and an articulation head 24, which are functionally similar to the implantation body 11 and the articulation head 14 of the humeral component 10 respectively. The implantation body 21 defines an implantation axis X21 that is similar to the implantation axis X11 for the implantation body 12 of the humeral component 10. Moreover, the implantation body 21 includes a peripheral part 22 and a central core 23, which are functionally similar to the peripheral part 12 and the central core 13 of the implantation body 11 respectively.

The peripheral part 22 of the implantation body 21 is distinguished by its structure, in the sense that the peripheral part 22 comprises or even consists of an elastic shell 22A and spacers 22B that mechanically connect the elastic shell 22A to the central core 23. The elastic shell 22A has a deformation capability that is varied along and around the implantation axis X21 depending on preoperative local bone density and quantity of the cancellous bone internal layer H2 of the humerus H. Upon implantation of the implantation body 21 in the humerus H, the elastic shell 22A applies on the cancellous bone internal layer H2 stresses resulting at least from the elastic material, the shape and the thickness of the elastic shell 22A and from the action of the spacers 22B on the elastic shell 22A, so that the peripheral part 22 can compact this layer H2 with a predicted compaction rate and/or value that is varied along and around the implantation axis X21 in accordance with the aforesaid planned manner with which the various portions of this layer H2 are desired to be compacted, as explained previously.

In practice, the peripheral part 22 may be manufactured by additive manufacturing processes.

Turning now to FIG. 4, a humeral component 30 is considered as a third embodiment of a humeral component produced by the method described above.

The humeral component 30 comprises an implantation body 31 and an articulation head 34, which are functionally similar to the implantation body 11 and the articulation head 14 of the humeral component 10 respectively. The implantation body 31 defines an implantation axis X31 that is similar to the implantation axis X11 for the implantation body 12 of the humeral component 10. Moreover, the implantation body 31 includes a peripheral part 32 and a central core 33, which are functionally similar to the peripheral part 12 and the central core 13 of the implantation body 11 respectively.

The peripheral part 32 is distinguished by its structure, in the sense that the peripheral part 32 comprises or even consists in an intermediate, soft undercoat 32A, which covers the central core 33, and an external, frangible coating 32B, which overlays the undercoat 32. Both undercoat 32A and coating 32B have a shape adaptability, that is to say that they can each adapt their shape upon implantation of the humeral component 30, the shape adaptability for each of the undercoat 32A and the coating 32B being varied along and around the implantation axis X31 depending on preoperative local bone density and quantity of the cancellous bone internal layer H2. However, these two shape adaptabilities are implemented by two different basic mechanisms: the undercoat 32A is conformable by elastic deformation whereas the coating 32B is conformable by surface breaking, on the understanding that the material of the coating 32B is bioabsorbable so that the detached particles from the coating 32B are absorbed by the tissues of the patient. This bioabsorbable material may be ceramic type and is for example a mixture of hydroxyapatite and tricalciumphosphate, or any brittle biological bone substitute. Thanks to the respective shape adaptability of the undercoat 32A and of the coating 32B, the peripheral part 32 of the implantation body 31 applies on the cancellous bone internal layer H2 some predetermined stresses upon implantation of the implantation body 31 in the humerus H, so that this peripheral part 32 can compact this layer H2 with a predicted compaction rate and/or value that is varied along and around the implantation axis X31 in accordance with the aforesaid planned manner with which the various portions of this layer H2 are desired to be compacted as explained previously.

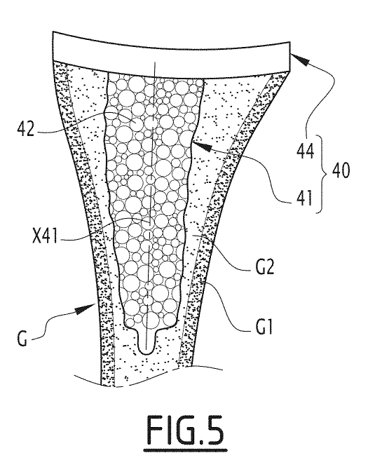

All of the above considerations relevant to the humerus of a patient and to producing a humeral component for this humerus are applicable to the glenoid of a patient and to produce a glenoid component for this glenoid. For example, FIG. 5 shows a glenoid G of a patient, which has a cortical bone external layer G1 and a cancellous bone internal layer G2 and which is prosthesized with a glenoid component 40. In the same way as explained previously for the humeral components 10, 20 and 30, the glenoid component 40 comprises an implantation body 41 that defines an implantation axis X41 and that is designed to compact the cancellous bone internal layer G2 in a planned manner that is differentiated along and around the implantation axis X41 depending on preoperative local bone density and quantity of this cancellous bone internal layer G2 when the glenoid component 40 is implanted at the glenoid G. In FIG. 5, the implantation body 41 is received within the glenoid G and the cancellous bone internal layer G2 of the glenoid G is compacted by the implantation body 41 in the aforesaid planned manner.

As for the implantation bodies 11, 21 and 31 of the humeral components 10, 20 and 30, the implantation body 41 may include a peripheral part 42 that is intended to be in contact with the cancellous bone internal layer G2, being able to apply stresses on this layer G2 by compacting the latter with a predicted compaction rate and/or value that is varied along and around the implantation axis X41 in accordance with the aforesaid planned manner with which the various portions of the layer G2 are desired to be compacted. Various embodiments for the implantation body 41 and for its peripheral part 42 may be considered. In FIG. 5, the peripheral part 42 is made of a porous structure as the one contemplated for the peripheral part 12 of the implantation body 11 of the humeral component 10. In alternative, the peripheral part 42 may correspond to one of the peripheral parts 22 and 32 of the implantation bodies 21 and 31.

In the embodiment of FIG. 5, the glenoid component 40 further comprises an articulation cup 44 which is functionally similar to the articulation heads 14, 24 and 34 of the humeral components 10, 20 and 30, except that an articulation surface of the articulation cup 44 is intended to articulate either with a humeral prosthetic component implanted at the humerus of the patient, such as one of the humeral components 10, 20 and 30, or directly with the head of the humerus of the patient, in both cases so as to restore shoulder joint.

The embodiment shown in FIG. 5 also illustrates that a central support such as the central cores 13, 23 and 33 disclosed above, may be omitted for the implantation body, as shown for the implantation body 41 in FIG. 5.

What is claimed is:

1. A shoulder prosthesis component for a patient,
comprising an implantation body, which defines an implantation axis and which is designed to be received within a bone of the patient that includes a cortical bone external layer and a cancellous bone internal layer, and to compact therein the cancellous bone internal layer in a planned manner that is differentiated along and around the implantation axis depending on preoperative local bone density and quantity of the cancellous bone internal layer.

2. The shoulder prosthesis component of claim 1, wherein the implantation body includes a peripheral part that is shaped and structured to compact the cancellous bone internal layer in said planned manner.

3. The shoulder prosthesis component of claim 2, wherein the peripheral part is also shaped and structured to be deformed while compacting the cancellous bone internal layer in said planned manner.

4. The shoulder prosthesis component of claim 2, wherein the peripheral part comprises a porous structure which has a porosity that is varied along and around the implantation axis depending on preoperative local bone density and quantity of the cancellous bone internal layer.

5. The shoulder prosthesis component of claim 2, wherein the implantation body further includes a central core that is more rigid than the peripheral part.

6. The shoulder prosthesis component of claim 5, wherein the peripheral part comprises an elastic shell, which is supported by the central core by means of spacers and which has a deformation capability that is varied along and around the implantation axis depending on preoperative local bone density and quantity of the cancellous bone internal layer.

7. The shoulder prosthesis component of claim 5, wherein the peripheral part comprises:
an intermediate, soft undercoat, which covers the central core and which has a shape adaptability that is implemented by deformation and that is varied along and around the implantation axis depending on preoperative local bone density and quantity of the cancellous bone internal layer, and
an external, frangible coating, which overlays the undercoat and which has a shape adaptability that is implemented by breaking and that is varied along and around the implantation axis depending on preoperative local bone density and quantity of the cancellous bone internal layer.

8. The shoulder prosthesis component of claim 1, wherein the shoulder prosthesis component further comprises an articulation member, which is provided with an articulation surface and which is provided to be coupled with the implantation body.

9. The shoulder prosthesis component of claim 1, wherein the shoulder prosthesis component is a humeral component, the implantation body being designed for the humerus of the patient.

10. The shoulder prosthesis component of claim 1, wherein the shoulder prosthesis component is a glenoid component, the implantation body being designed for the glenoid of the patient.

11. A method for producing a shoulder prosthesis component for a patient, the method comprising:
a data providing step, in which a bone of the patient that includes a cortical bone external layer and a cancellous bone internal layer is characterized by patient bone data representative of preoperative local bone density and quantity of the cancellous bone internal layer,
a design step, in which an implantation body of the shoulder prosthesis component to be produced is designed from the patient bone data so that the bone as prosthesized with the shoulder prosthesis component to be produced receives inwardly the implantation body and has its cancellous bone internal layer that is compacted in a planned manner that is differentiated along and around an implantation axis of the implantation body depending on preoperative local bone density and quantity of the cancellous bone internal layer, and
a manufacturing step, in which the implantation body designed at the design step is manufactured.

12. The method of claim 11, wherein the design step includes:
defining rules which are based on the patient bone data and which relate preoperative local bone density and quantity of the cancellous bone internal layer to a desired compacted condition for the cancellous bone internal layer of the bone as prosthesized with the shoulder prosthesis component to be produced, and
using the rules to design the implantation body.

13. The method of claim 11, wherein the data providing step includes performing a CT scan of the bone and segmenting CT images of the CT scan into the patient bone data.

14. The method of claim 11, wherein the manufacturing step includes using an additive manufacturing process.

15. The method of claim 11, wherein the method further comprises coupling the implantation body manufactured at the manufacturing step with an articulation member of the shoulder prosthesis component, the articulation member being provided with an articulation surface.
